(19) United States
(12) Patent Application Publication
(10) Pub. No.: US 2019/0240193 A1
(43) Pub. Date: Aug. 8, 2019

(54) COMPOSITIONS COMPRISING CROMOGLICIC ACID FOR THE TREATMENT OF DERMATITIS

(71) Applicants: Diater Laboratorio De Diagnóstico Y Aplicaciones Terapéuticas, S.A., Leganés, Madrid (ES); Farmalider S.A., Alcobendas, Madrid (ES)

(72) Inventors: Ricardo Palacios Peláez, Leganés, Madrid (ES); Javier Alcover Díaz, Leganés, Madrid (ES); David Rodríguez Gil, Leganés, Madrid (ES); Fernando Pineda De La Losa, Leganés, Madrid (ES); Concepción Tiana Ferrer, Alcobendas, Madrid (ES); Laura Fernández Lorenzana, Alcobendas, Madrid (ES); Jose Ángel Sánchez García, Alcobendas, Madrid (ES); Marta Vicario De La Torre, Alcobendas, Madrid (ES)

(21) Appl. No.: 16/314,971

(22) PCT Filed: Jul. 6, 2017
(86) PCT No.: PCT/ES2017/070490

§ 371 Date: Jan. 3, 2019

(30) Foreign Application Priority Data
Jul. 7, 2016 (ES) P201630928

(51) Int. Cl. A61K 31/352; A61K 36/47; A61K 9/06; A61K 9/107; A61K 31/164; A61P 17/04; A61K 9/00; A61K 47/10; A61K 45/06; A61K 8/49

(52) U.S. Cl. CPC A61K 31/352; A61K 36/47; A61K 9/06; A61K 9/107; A61K 8/498; A61P 17/04; A61K 9/0014; A61K 47/10; A61K 45/06; A61K 31/164

(57) ABSTRACT

The present invention relates to a combination comprising cromoglicic acid or derivatives thereof and to dermatological compositions comprising said composition. The invention also relates to the use of said combination or composition for the treatment of dermatitis.

### FIELD OF THE INVENTION

The present invention relates to the dermatological use of new compositions comprising cromoglicic acid or derivatives thereof.

### BACKGROUND OF THE INVENTION

Dermatitis is an acute or chronic inflammatory reaction of the skin that may have different degrees of severity. It is a disease in which both constitutional factors, for example greater immune sensitivity and genetic alterations, and a multitude of exposure factors that induce or help to maintain and exacerbate the symptoms.

Generally, at the onset of dermatitis oedematous erythema can be observed, followed by erythematous injuries with papules and serous papules, followed by the formation of vesicles, pustules, erosions, scabs and peeling. When the dermatitis is acute, these conditions are usually recurrent. When the dermatitis becomes chronic, thickening, lichenification and pigmentations of the skin are additionally observed, often associated with itching.

Dermatitis is usually divided into different types, including atopic dermatitis, contact dermatitis, seborrheic dermatitis, eczemas, psoriasis and other.

The current most common treatments for dermatitis involve identifying and avoiding factors that may induce or exacerbate the inflammation, as well as proper skin care, nearly always in combination with the use of adequate drugs for treating the symptoms of the disease. Among said drugs, steroid formulations are commonly used. However, despite their proven clinical effectiveness, steroid-based formulations, such as for example topical corticosteroid preparations, have many side effects such as excessive thinning of the skin, atrophy, so-called “moon face” caused, inter alia, by the deposition of grease on the skin, reddened skin, hirsutism and stretch marks. Additionally, when the patient is exposed to a long-term use of steroids, the body usually becomes resistant to them and there are even cases in which the dermatitis symptoms flare up more aggressively.

Different types of non-steroid dermatitis therapies are currently used and known. In particular, the use of cromoglicic acid or derivatives thereof is suggested in the state of the art.

Cromoglicic acid is a mastocyte stabiliser and is commonly marketed in its sodium salt (sodium cromoglycate) form. The most frequent uses of this drug are in the treatment of asthma and conjunctivitis.

WO 99/60997 A1 describes sodium cromoglycate formulations that include an amphoteric surfactant and an alkoxylated cetyl alcohol as essential ingredients for the treatment of atopic dermatitis.

EP 0084190 A2 describes a composition comprising cromoglicic acid, or salts thereof, as well as hydrophilic macromolecular material. The composition is useful in the treatment of skin wounds and injuries that produce secretions, such as those caused by dermatitis.

EP 1040826 A1 discloses cromoglicic acid esters that are useful in the treatment of dermatitis in which an allergic reaction takes place.

Despite the existence of the different formulations based on cromoglicic acid suggested in the state of the art for the treatment of dermatitis, there is a continuous need to develop new alternative dermatological compositions that enrich the arsenal of available treatments.

### COMPENDIUM OF THE INVENTION

The authors of the present invention have now discovered a combination of ingredients comprising cromoglicic acid or a derivative thereof and that is appropriate for treating dermatitis. It has also been discovered that the ingredients of the combination act in an unexpectedly synergistic manner, thereby offering a particularly effective and/or safe treatment against dermatitis.

Therefore, in a first aspect the invention is aimed at a combination comprising:

a) a compound of formula (I)

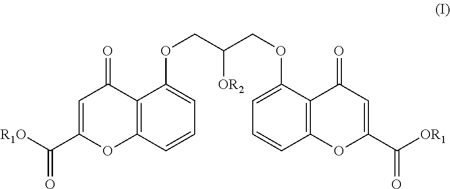

wherein
each R₁, independently, is selected from H or, together with the —O—C(O)— group whereto it is bonded, forms an ester or carboxylic anhydride group; and
R₂ is selected from H or, together with the oxygen atom whereto it is bonded, forms an ether, ester, carbamate or carbonate group;
or a salt or solvate thereof;

b) Croton lechleri resin; and

c) panthenol.

In a second aspect, the invention relates to a pharmaceutical composition comprising the combination of the first inventive aspect and a pharmaceutically acceptable carrier.

Another aspect of the present invention is aimed at the combination of the first aspect of the invention or at the combination of the second aspect of the invention, for use as a drug, particularly for use in the treatment or prophylaxis of dermatitis.

Also forming part of the object of the invention is the use of the combination of the first aspect of the invention, or of the composition of the second aspect of the invention, for preparing a drug, particularly a drug intended for the treatment or prophylaxis of dermatitis.

Also, the object of the present invention is a method for treating dermatitis comprising: a) providing a subject requiring a dermatitis treatment or prophylaxis; and b) administering the combination of the first aspect of the invention or the composition of the second aspect of the invention to said subject.

Also, the object of the present invention is the cosmetic (non-therapeutic) use of the combination of the first aspect of the invention or of the composition of the second aspect of the invention in the treatment of the skin to counteract the effects of dermatitis.

### BRIEF DESCRIPTION OF THE DRAWINGS

FIG. 1 shows the changes in ear thickness during the sensitisation phase and during the subsequent treatment phase of the assay described in Example 2. Each entry corresponds to a group of mice and represents the average ear thickness considered based on the ear thickness of all the ears collected in said group;

FIG. 2 shows all the changes in ear thickness during the sensitisation phase and during the subsequent treatment phase of the trial described in Example 3. Each entry corresponds to a group of mice and represents the average ear thickness considered based on the ear thickness of all the ears collected in said group;

FIG. 3 shows the changes in PAR2 levels in mouse ear tissue after treating with the compositions of the present invention;

FIG. 4 shows the changes in TRVP4 levels in mouse ear tissue after treating with the compositions of the present invention; and

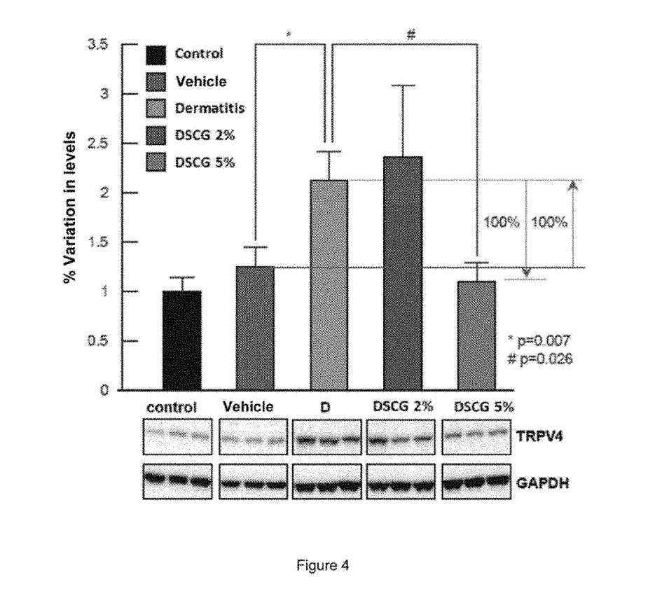

FIG. 5 shows the changes in TNF-alpha levels in mouse ear tissue after treating with the compositions of the present invention.

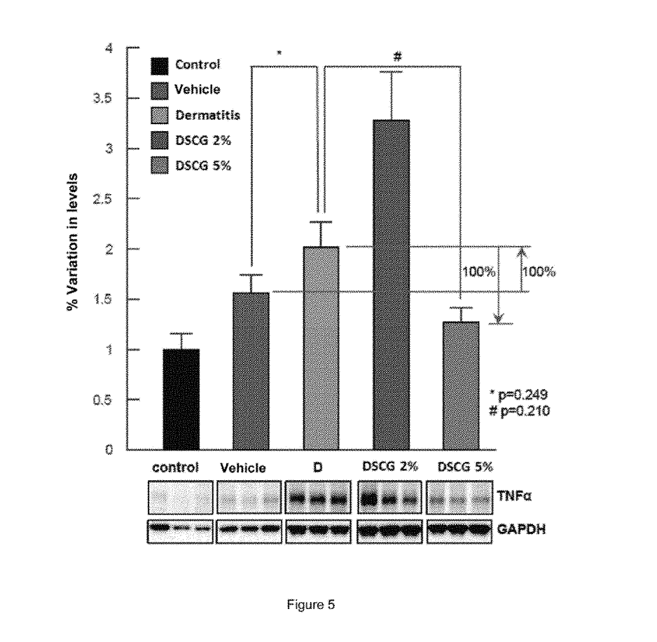

### DETAILED DESCRIPTION OF THE INVENTION

An object of the present invention is a combination comprising:

a) A compound of formula (I)

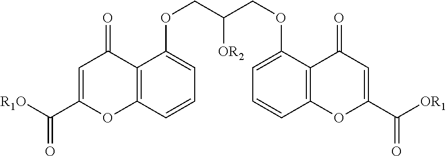

wherein
each R₁, independently, is selected from H or, together with the —O—C(O)— group whereto it is bonded, forms an ester or carboxylic anhydride group; and
R₂ is selected from H or, together with the oxygen atom whereto it is bonded, forms an ether, ester, carbamate or carbonate group;
or a salt or solvate thereof;

b) Croton lechleri resin; and

c) panthenol.

### Compound of Formula (I)

The compound of formula (I) comprised in the combination of the present invention is cromoglicic acid or a derivative thereof. Cromoglicic acid (IUPAC name: 5,5′-(2-hydroxypropane-1.3-diyl)bis(oxy)bis(4-oxo-4H-cremona-2-carboxylic acid) is a mastocyte stabiliser typically known for its use in the treatment of asthma and conjunctivitis. The formula of the compound is as follows:

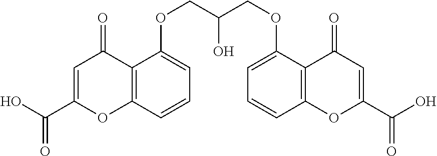 [SMILES: O=C(O)c1cc(=O)c2c(OCC(O)COc3cccc4oc(C(=O)O)cc(=O)c34)cccc2o1]

The compounds of formula (I) can be obtained from commercial sources or prepared using chemical synthesis methods described in the state of the art or variations thereof that are commonly known to a person skilled in the art. For example, the compounds of formula (I) can be prepared as described in patent application EP 1040826 A1.

In one embodiment, R₁, independently or simultaneously, is selected from H or, together with the —O—C(O)— whereto it is bonded, forms an ester group; and R₂ is selected from H or, together with the oxygen atom whereto it is bonded, forms an ether or ester group.

In one embodiment, in any of the previous embodiments in the compound of formula (I) or the salt or solvate thereof, those mentioned in the ether, ester, carboxylic anhydride, carbamate and carbonate group are as defined below.

In a particular embodiment, R₁ is H or X, wherein X represents a straight-chain or branched alkyl group, preferably represents a straight-chain or branched C₁₋₁₂ alkyl group, more preferably represents a straight-chain or branched C₁₋₆ alkyl group. In a preferred embodiment, X is methyl, ethyl, propyl or isopropyl; or straight-chain or branched butyl (for example, branched butyl is terc-butyl). In a particularly preferred embodiment, X is methyl or ethyl, preferably ethyl.

In another embodiment, R₁ is H or a group

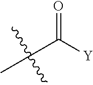

wherein Y is H or X as defined earlier. Preferably, Y is X as defined earlier. Preferably, Y is methyl.

In one embodiment, R₂ is H or X, wherein X represents a straight-chain or branched alkyl group, preferably represents a straight-chain or branched C₁₋₁₂ alkyl group, more preferably represents a straight-chain or branched C₁₋₆ alkyl group. In a preferred embodiment, X is methyl, ethyl, propyl or isopropyl; or straight-chain or branched butyl (for example, branched butyl is terc-butyl). In a particularly preferred embodiment, X is methyl or ethyl, preferably ethyl.

In another embodiment, R₂ is H or a group

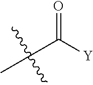

wherein Y is as defined earlier.

In another embodiment, R₂ is H or a group

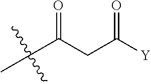

wherein Y is as defined earlier.

In another embodiment, R₂ is H or a group

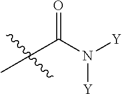

wherein each Y, independently or simultaneously, is as defined earlier.

In another embodiment, R₂ is H or a group

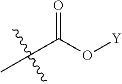

wherein Y is as defined earlier.

In a specific embodiment, R₁ is H or X, preferably ethyl, and R₂ is

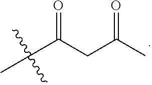 [SMILES: *C(=O)CC(C)=O]

In another specific embodiment, R₁ is H or X, wherein X is as defined earlier, and R₂ is H.

In an even more preferred embodiment, R₁ and R₂ are H, i.e. the compound of formula (I) is cromoglicic acid.

In the context of the present invention, the salt of the compound of formula (I), preferably of cromoglicic acid, is a pharmaceutically acceptable salt. Pharmaceutically acceptable salt is understood to be any salt that is physiologically tolerated when used properly for a treatment, applied or used, particularly, in humans and/or mammals. Preferably, the pharmaceutically acceptable salt is a dermatologically acceptable salt, i.e. it is a salt for applying on the skin which does not have toxicity, incompatibility, irritation, allergic response or similar problems.

Preferably, the salt is a salt wherein the compound of formula (I) forms the anion, preferably, and where applicable, by deprotonation of at least one carboxylic and/or hydroxyl group, and the counter cation is an alkali or alkaline earth metal. More preferably, the counter cation is sodium or potassium. Even more preferably, the counter cation is sodium.

Preferably, the salt is a cromoglicic acid salt wherein one or both carboxylic groups and/or the secondary hydroxyl group are in a deprotonated state and the counter cation is an alkali or alkaline earth metal, or an ammonium cation. Preferably, the counter cation is an alkali metal. More preferably, the counter cation is sodium or potassium. Even more preferably, the counter cation is sodium.

More preferably, the salt is a cromoglicic acid salt wherein the carboxylic groups are in a deprotonated state and the counter cation is an alkali or alkaline earth metal, or an ammonium cation. Preferably, the counter cation is an alkali metal. More preferably, the counter cation is sodium or potassium. Even more preferably, the counter cation is sodium.

In a particularly preferred embodiment, the compound of formula (I) is sodium cromoglycate, which can be monosodium or disodium, i.e. the cromoglicic acid salt wherein one or both carboxylic groups, respectively, is/are in a deprotonated state, and the counter cation (of each deprotonated carboxylic group) is sodium. Preferably, the compound of formula (I) is disodium cromoglycate (DSCG).

The aforementioned salts can be obtained from commercial sources or prepared following widely known methods in the field of the invention. For example, the salts wherein the counter cation of the carboxylic groups is an alkali or alkaline earth metal can be prepared by reacting the cromoglicic acid with alkali or alkaline earth metal hydroxides or alkoxides in an appropriate solvent.

In the context of the present invention, the solvate of the compound of formula (I), preferably of the cromoglicic acid, even more preferably of the disodium cromoglycate, is any form wherein said compound is bonded to another molecule (normally a polar solvent) by means of a non-covalent bond, especially including hydrates and alcoholates such as, for example, methanolate. A preferred solvate is the hydrate.

Croton lechleri Resin

Croton lechleri is the scientific name of the so-called “Dragon's Blood” tree, which belongs to the Euphorbiaceae family. A resin is extracted from said tree whose most active components are proanthocyanidins and the taspine alkaloid. Other components of the resin are lignans, polyunsaturated fatty acids, pigments and flavonoids.

The paper by K. Jones, Review of Dragon's Blood (Croton lechleri)—A South American Tree Sap in the Treatment of Diarrhea, Inflammation, Insect Bites, Viral Infections and Wounds: Traditional Uses to Clinical Research, J. Altern. Complem. Med., 2003, 9(6), 877-896, describes the properties and traditional applications of the resin of C. lechleri.

C. lechleri resin can be found commercially, for example in the form of hydroglycolic solution (water and propylene glycol), under the name Dragon's Blood, from the company Cobiosa, wherein the resin content is comprised between 1% and 5% by weight.

Therefore, in one embodiment, the C. lechleri resin comprised in the combination of the invention is a hydroglycolic solution, preferably from water and propylene glycol. Preferably, this hydroglycolic solution comprises the resin in content by weight between 1% and 5%.

### Panthenol

Panthenol is a compound belonging to the vitamin B group and is transformed into pantothenic acid (vitamin B₅) on the skin, due to which it is also called provitamin B₅.

Said compound is well known in the cosmetic industry, since it has been used for years as a skin moisturiser.

Panthenol is a chemical compound which has a chiral carbon atom, due to which it exists in two enantiomeric forms, of which only D-panthenol (dexpanthenol) is biologically active. However, both enantiomers have moisturising activity and both D-panthenol and the racemic mixture that includes D-panthenol and L-panthenol can be used in cosmetic compositions.

In a preferred embodiment, in the combination of the invention, panthenol is selected from D-panthenol and the racemic mixture, and D-panthenol is preferably used.

### Combinations

In one embodiment, the proportion between Croton lechleri resin and panthenol is comprised between 1:500 and 1:2, expressed as weight:weight, preferably between 1:200 and 1.10, especially between 1.100 and 1.20, with special preference between 1.50 an 1:30, and in particular between 1:50 and 1:40, more particularly between 1:45 and 1:44.

The combination of the weight of Croton lechleri resin and panthenol forms weight B. In a preferred embodiment, the proportion by weight between the weight of the compound of formula (I) (or a salt or solvate thereof), preferably of cromoglicic acid (or a salt or solvate thereof), preferably of disodium cromoglycate (or a solvate thereof), and weight B is 2.5:2.045 or higher (understanding by higher that the weight of the compound of formula (I) increases or weight B decreases), 4.5:2.045 or higher; or 4.9:2.045 or higher. More specifically, the proportion is between 2.5:2.045 and 10:2.045; between 4.5:2.045 and 10:2.045; or between 4.9:2.045 and 10:2.045. More specifically, the proportion is between 2.5:2.045 and 5.5:2.045; between 4.5:2.045 and 5.5:2.045; or between 4.9:2.045 and 5.5:2.045. More specifically, the proportion is between 2.5:2.045 and 5.1:2.045; between 4.5:2.045 and 5.1:2.045; or between 4.9:2.045 and 5.1:2.045.

In a preferred embodiment, the proportion by weight between the weight of the compound of formula (I) (or a salt or solvate thereof), preferably of cromoglicic acid (or a salt or solvate thereof), preferably of disodium cromoglycate (or a solvate thereof); and the weight of panthenol is 2.5:2.000 or higher (understanding by higher that the weight of the compound of formula (I) increases or the weight of panthenol decreases), 4.5:2.000 or higher; or 4.9:2.000 or higher. More specifically, the proportion is between 2.5:2.000 and 10:2.000; between 4.5:2.000 and 10:2.000; or between 4.9:2.000 and 10:2.000. More specifically, the proportion is between 2.5:2.000 and 5.5:2.000; between 4.5:2.000 and 5.5:2.000; or between 4.9:2.000 and 5.5:2.000. More specifically, the proportion is between 2.5:2.000 and 5.1:2.000; between 4.5:2.000 and 5.1:2.000; or between 4.9:2.000 and 5.1:2.000.

In a preferred embodiment, the proportion by weight between the weight of the compound of formula (I) (or a salt or solvate thereof), preferably of cromoglicic acid (or a salt or solvate thereof), preferably of disodium cromoglycate (or a solvate thereof); and the weight of Croton lechleri resin is 2.5:0.045 or higher (understanding by higher that the weight of the compound of formula (I) increases or the weight of Croton lechleri resin decreases), 4.5:0.045 or higher; or 4.9:0.045 or higher. More specifically, the proportion is between 2.5:0.045 and 10:0.045; between 4.5:0.045 and 10:0.045; or between 4.9:0.045 and 10:0.045. More specifically, the proportion is between 2.5:2.045 and 5.5:2.045; between 4.5:2.045 and 5.5:2.045; or between 4.9:2.045 and 5.5:2.045. More specifically, the proportion is between 2.5:2.045 and 5.1:2.045; between 4.5:2.045 and 5.1:2.045; or between 4.9:2.045 and 5.1:2.045.

In a particular embodiment, the combination of the invention consists essentially of:
a) at least one compound of formula (I) (or a salt or solvate thereof), preferably cromoglicic acid (or a salt or solvate thereof), preferably disodium cromoglycate (or a solvate thereof);
b) Croton lechleri resin;
c) panthenol.

According to this embodiment, the combination cannot comprise additional ingredients that are active ingredients against dermatitis.

In a particular embodiment, the combination of the invention consists of:
a) at least one compound of formula (I) (or a salt or solvate thereof), preferably cromoglicic acid (or a salt or solvate thereof), preferably disodium cromoglycate (or a solvate thereof);
b) Croton lechleri resin;
c) panthenol.

The combinations of the present invention may comprise additional ingredients.

In a particular embodiment, the combination of the invention comprises aloe vera gel. Aloe vera gel is obtained from the succulent plant Aloe barbadensis of the Asphodelaceae family. The leaves of said plant are composed of three layers: an outer coriaceous protection, a fibrous layer beneath it and a gelatinous core where it stores its water reserves and which is used in the preparation of dermatological products and cosmetics. Aloe vera gel extracted from the plant with solid content of approximately 0.5% by weight can be found on the market under the name gel 1:1. It can also be found in the form of concentrated gel wherefrom part of the water has been extracted. For example, the concentrated gel 2:1 has a solid content of approximately 1% by weight; the concentrated gel 10:1 has a solid content of approximately 5% by weight; the concentrated gel 40:1 has a solid content of approximately 20% by weight. Atomised products 100:1 and 200:1 can also be found, which make it possible to reconstitute the original gel on mixing 1 part thereof and 99 parts of water or 1 part and 199 parts of water, respectively.

In the context of the invention, a concentrated aloe vera gel 10:1 is used, such that the combination of 1 part of said gel with 9 parts of water leads to the original gel of the plant.

In another particular embodiment, the combination of the invention comprises rosehip oil.

Rosehip is a plant belonging to the Rosaceae family, which encompasses three species: Rosa moschata, Rosa canina and Rosa rubiginosa. It is a bush with thin, flexible and very thorny branches which can exceed 2 metres in height. It is currently extended across various geographical regions, especially those with temperate climates. Oil is extracted from the seeds contained in its fruit.

In one form of embodiment, the present invention relates to a combination as defined earlier, which additionally comprises:
d) aloe vera gel and/or Rosehip oil.

In a particular embodiment, the combination of the invention consists essentially of:
a) at least one compound of formula (I) (or a salt or solvate thereof), preferably cromoglicic acid (or a salt or solvate thereof), preferably disodium cromoglycate (or a solvate thereof);
b) Croton lechleri resin;
c) panthenol; and
d) aloe vera gel and/or rosehip oil.

According to this embodiment, the combination cannot comprise additional ingredients that are active ingredients against dermatitis.

In a particular embodiment, the combination of the invention consists of:
a) at least one compound of formula (I) (or a salt or solvate thereof), preferably cromoglicic acid (or a salt or solvate thereof), preferably disodium cromoglycate (or a solvate thereof);
b) Croton lechleri resin;
c) panthenol; and
d) aloe vera gel and/or rosehip oil.

In a preferred embodiment, the proportion by weight between the weight of the compound of formula (I) (or a salt or solvate thereof), preferably of cromoglicic acid (or a salt or solvate thereof), preferably of disodium cromoglycate (or a solvate thereof); and the weight of aloe vera gel (preferably concentrated 10:1) is 2.5:3.000 or higher (understanding by higher that the weight of the compound of formula (I) increases or the weight of the aloe vera gel decreases), 4.5:3.000 or higher; or 4.9:3.000 or higher. More specifically, the proportion is between 2.5:3.000 and 10:3.000; between 4.5:3.000 and 10:3.000; or between 4.9:3.000 and 10:3.000. More specifically, the proportion is between 2.5:3.000 and 5.5:3.000; between 4.5:3.000 and 5.5:3.000; or between 4.9:3.000 and 5.5:3.000. More specifically, the proportion is between 2.5:3.000 and 5.1:3.000; between 4.5:3.000 and 5.1:3.000; or between 4.9:3.000 and 5.1:3.000.

In a preferred embodiment, the proportion by weight between the weight of the compound of formula (I) (or a salt or solvate thereof), preferably of cromoglicic acid (or a salt or solvate thereof), preferably of disodium cromoglicic (or a solvate thereof); and the weight of rosehip oil is 2.5:0.500 or higher (understanding by higher that the weight of the compound of formula (I) increases or the weight of the rosehip oil decreases); 4.5:0.500 or higher; or 4.9:0.500 or higher. More specifically, the proportion is between 2.5:0.500 and 10:0.500; between 4.5:0.500 and 10:0.500; or between 4.9:0.500 and 10:0.500. More specifically, the proportion is between 2.5:2.000 and 5.5:0.500; between 4.5:0.500 and 5.5:0.500; or between 4.9:0.500 and 5.5:0.500. More specifically, the proportion is between 2.5:0.500 and 5.1:0.500; between 4.5:0.500 and 5.10.500; or between 4.9:0.500 and 5.1:0.500.

### Compositions

Also, the object of the invention is a pharmaceutical composition comprising the combination of the invention and a pharmaceutically acceptable carrier. Pharmaceutically acceptable carrier is understood to be a carrier that is physiologically tolerated when used properly for a treatment, applied or used, particularly, in humans and/or mammals, i.e. a carrier that does not have toxicity, incompatibility, irritation, allergic response or similar problems, and does not have incompatibilities with the ingredients of the combination of the invention. Preferably, the pharmaceutical composition is a dermatological composition and the pharmaceutically acceptable carrier is a dermatologically acceptable carrier. Dermatological composition is understood to be a pharmaceutical composition suitable for treating and/or preventing skin conditions, and dermatologically acceptable carrier is understood to be a suitable carrier for applying to the skin that does not have toxicity, irritation, allergic response or similar problems for the skin.

In a preferred embodiment, the compositions of the invention comprise:
between 4.7% and 27% by weight of the combination of the invention; and
between 95.3% and 73% by weight of the pharmaceutically acceptable carrier;
   adjusting the percentages of the components such that the balance is 100%.

Preferably, the weight of the combination of the invention with respect to the total weight of the composition is between 5% and 20%, especially between 7% and 15%, particularly between 10% and 11% and even more particularly between 10.5% and 10.6%.

In one embodiment, the weight of the compound of formula (I) (or a salt or solvate thereof), preferably cromoglicic acid (or a salt or solvate thereof), preferably disodium cromoglycate (or a solvate thereof), is between 0.01%, 0.1%, 1% and 30% with respect to the total weight of the composition. In another embodiment, it is between 0.01%, 0.1%, 1% and 20%. In another embodiment it is between 0.01%, 0.1%, 1% and 10%.

In a preferred embodiment, the weight of the compound of formula (I) (or a salt or solvate thereof), preferably cromoglicic acid (or a salt or solvate thereof), preferably disodium cromoglycate (or a solvate thereof), is 2.5%, 3%, 3.5%, 4% or 4.5% or higher with respect to the total weight of the composition. In another preferred embodiment, the weight is between 2.5%, 3%, 3.5%, 4% or 4.5% and 10% with respect to the total weight of the composition. In another more particular embodiment, the weight is between 2.5%, 3%, 3.5%, 4% or 4.5% and 8%. In another more particular embodiment, the weight is between 2.5%, 3%, 3.5%, 4% or 4.5% and 7%. In another more particular embodiment, the weight is between 2.5%, 3%, 3.5%, 4% or 4.5% and 6%. In another more particular embodiment, the weight is between 2.5%, 3%, 3.5%, 4% or 4.5% and 5.5%. In another more particular embodiment, the weight is between 2.5%, 3%, 3.5%, 4% or 4.5% and 5.1%. In another more particular embodiment, the weight is between 2.5%, 3%, 3.5%, 4% or 4.5% and 5%. In a more particular embodiment, the weight is between 4.5% and 5.5%. In an even more particular embodiment, the weight is between 4.9% and 5.1%.

As mentioned earlier, the weight of Croton lechleri resin plus the weight of panthenol, preferably D-panthenol, is weight B. In one embodiment, weight B is 0.2-12% with respect to the total weight of the composition, preferably 1-9%, more preferably 1-3%, even more preferably 1.5-2.5%, even more preferably 2.0-2.1%. For each of these weights, the weight ratio between Croton lechleri resin and panthenol, preferably D-panthenol, is comprised between 1:500 and 1:2, expressed as weight:weight, preferably between 1:200 and 1.10, especially between 1.500 and 1:20, with special preference between 1.50 and 1:30, and in particular between 1:50 and 1:40, more particularly between 1:45 and 1:44.

In another embodiment, the weight of the aloe vera gel, preferably of the aloe vera gel concentrate 10:1, ranges between 2% and 4%, preferably between 2.5% and 3.5%, especially between 2.9% and 3.1% with respect to the total weight of the composition, or is 3.0%.

In another embodiment, the weight of the rosehip oil ranges between 0.01% and 1%, preferably between 0.1% and 1%, especially between 0.3% and 0.8%, and in particular between 0.4% and 0.6%, more particularly between 0.45% and 5.5%, especially particularly between 0.49% and 0.51% with respect to the total weight of the composition, or is 0.50%.

Preferably, the ingredients of the combination of the invention are dissolved, emulsified, dispersed or suspended in the pharmaceutically acceptable carrier. Said carrier is selected from water, a water-soluble non-aqueous carrier, for example ethanol, isopropanol, and a water-soluble non-aqueous carrier, such as for example paraffin wax. Preferably, the compositions of the invention include water as a pharmaceutically acceptable carrier.

In other embodiments, the compositions of the invention also comprise at least one additional dermatological or cosmetic active ingredient.

In a particular embodiment, the weight of the total additional dermatological and cosmetic active ingredients is between 5% and 40% with respect to the total weight of the composition.

More particularly, the composition of the invention comprises:
between 4.7% and 27% by weight of the combination of the invention;
between 5% and 40% by weight of the additional dermatological and cosmetic active ingredients (i.e. the joint weight of the additional dermatological and cosmetic active ingredients); and
between 33% and 90.3% by weight of the pharmaceutically acceptable;
   adjusting the percentages of the components such that the balance is 100%.

In more specific embodiments, the weights of the combination of the invention are as defined earlier.

In a more specific embodiment, the weight of the additional dermatological or cosmetic active ingredients is comprised between 10% and 35%, in particular between 15% and 30%, especially between 20% and 25% with respect to the total weight of the composition.

In one embodiment, the additional dermatological active ingredient is a steroid, more preferably a corticosteroid. In a more particular embodiment, the corticosteroid is selected from prednisolone, hydrocortisone butyrate, dexamethasone valerate, betamethasone dipropionate, clobetasol propionate and clobetasone butyrate.

In another embodiment, said additional dermatological active ingredient is an antihistamine. In a more particular embodiment, the antihistamine is selected from diphenhydramine hydrochloride, mequitazine, prometazine hydrochloride and chlorpheniramine maleate.

In another embodiment, said additional dermatological active ingredient is an antiallergen. In a more particular embodiment, the antiallergen is selected from tranilast, ketotiphene fumarate, oxatomide, azelastine hydrochloride.

In another embodiment, said additional dermatological active ingredient is an antibacterial agent, preferably an antibacterial agent useful for treating acne. In a more particular embodiment, the antibacterial agent is clindomycin phosphate or tetracycline.

In one embodiment, said cosmetic ingredient is selected from at least: a humectant, emulsifying agent, emollient, a silicone, a chelating agent, preservative and a pH regulator.

Humectants are substances that moisturise and smooth the skin. The humectants, in the context of the present invention, can be selected, inter alia, from glycerine, propylene glycol, glycols, polyethylene glycols and mixtures thereof.

In one embodiment, the content of humectants is comprised between 5% and 10% by weight with respect to the total weight of the composition.

Emulsifying agents favour the formation of intimate mixtures of non-miscible liquids by alteration of the interfacial tension. The emulsifying agents, in the context of the present invention, can be selected, inter alia, from polysorbates, sorbitan esters, fatty alcohols, ethoxylated fatty alcohols, fatty acids and mixtures thereof such as beeswax.

In one embodiment, the content of emulsifying agents is comprised between 5% and 10% by weight with respect to the total weight of the composition.

Emollients contribute to optimising the organoleptic and dermatological properties of the composition. Emollients, in the context of the present invention, can be selected, inter alia, from alkanes and esters, such as glycerides, propylene glycol esters, alkyl esters, ethers, glycols and mixtures thereof.

The total amount of emollients is comprised between 8% and 12% by weight with respect to the total weight of the composition.

Silicones can also be used in the formulations of the present invention which, although they could be identified as a type of emollient, in the context of the present invention they are presented separately from the emollients. The silicones, in the context of the present invention, can be selected, inter alia, from poly(dialkylsiloxanes), poly(diarylsiloxanes) and poly(alkylarylsiloxanes), such as for example dimethicone, hexamethylcyclotrisiloxane, phenylmethicone, cyclomethicone, hexamethylcyclotrisiloxane or poly(methylphenylsiloxane).

The total amount of silicones is comprised between 1% and 3% by weight with respect to the total weight of the composition.

Chelating agents complex and neutralise metal ions that can affect the stability and/or appearance of the composition. Chelating agents can be monodentate and multidentate. The chelating agents, in the context of the present invention, can be selected, inter alia, from ethylenediaminatetracetic acid (EDTA), nitrilotriacetic acid (NTA), hydroxyethyl-ethylene-diamine-triacetic (HEEDTA), diethylene-triamine-pentetic acid (DTPA), diethanol-glycine (DEG), ethanoldiglycine (EDG), citric acid, phosphoric acid and tartaric acid, or their salts, and mixtures thereof.

The total amount of chelating agents is comprised between 0.01% and 1% by weight with respect to the total weight of the composition.

Preservatives stop or minimise the deterioration of the components of the formulation caused by the presence of different types of microorganisms. The preservatives, in the context of the present invention, can be selected, inter alia, from phenoxyethanol, tropolone, chlorphenesine, ethylhexylglycerine, isothiazolidone, diazolidinyl urea, imidazolidinyl urea and parabens, and mixtures thereof.

The total amount of preservatives is comprised between 0.5% and 1.5% by weight with respect to the total weight of the composition.

pH regulators establish and/or maintain the pH of the composition at the desired value. The pH regulators, in the context of the present invention, can be selected, inter alia, from citric acid, acetic acid, phosphoric acid, propionic acid, lactic acid, carbonic acid, ammonium/ammonia, sodium hydroxide and mixtures thereof.

The total amount of pH regulators is comprised between 0.01% and 1% by weight with respect to the total weight of the composition.

The compositions of the present invention contain water and a lipophile phase and are generally presented in the form of emulsions or dispersions, for example of the oil in water (O/W), water in oil (W/O) and multiple emulsions (W/O/W) type and PIT-type emulsions, as described in Spanish patent ES 2169908 T3, or as microemulsions. They can also be presented in the form of suspensions. Even more preferably, they are found in the form of oil in water-type (O/W) emulsions.

The compositions of the present invention are presented in different pharmaceutical forms, preferably dermatological, even more preferably dermatological forms for topical application, even more preferably dermatological forms applied topically to the skin. For example, the compositions are presented in the form of cream, pomade, ointment, balsam, lotion, milk, gel, foam, gelatin, etc.

When the compositions are prepared with low viscosity, they can also be applied by spraying, i.e. in these cases the compositions can be presented in spray form, for example as a spray or aerosol. In a preferred embodiment, the compositions are a sprayable emulsion.

The compositions can also be incorporated to sponges, tapes, patches, dressings or bandages. In a particular embodiment, the compositions are presented in patch form, preferably a transdermal patch.

Preferably, the compositions of the invention are found in the form of a cream, lotion, milk or sprayable emulsion. Even more preferably, they are found in the form of a cream.

### Preparation Method

The compositions of the invention can be prepared following conventional methods in the cosmetics/pharmaceutical industry for preparing lotions, milks, creams and sprayable emulsions. The preparation of emulsions is described, for example, in Remington's handbook: The Science and Practice of Pharmacy, 20th edition. Lippincott Williams & Wilkins, Philadelphia, 2000 [ISBN: 0-683-306472].

A method for preparing a composition of the invention can be, for example, the following.

Hydrophilic components such as water, glycerine and panthenol are weighed and melted in a reactor equipped with stirring equipment at a temperature comprised between 70° C. and 85° C., preferably between 75° C. and 85° C. The lipophilic components and emulsifiers are weighed and heated in another receptacle at a temperature comprised between 70° C. and 85° C., preferably between 75° C. and 80° C. Next, an emulsion is prepared from the lipophilic mass and the hydrophilic components with appropriate stirring. Lastly, the emulsion obtained is cooled to a temperature comprised between 35° C. and 45° C., preferably 40° C. (+/−2° C.).

Parallel to the emulsion method, in a different receptacle, water is heated to a temperature between 35° C. and 45° C., preferably 40° C. (+/−2° C.), after which the compound of formula (I), preferably cromoglicic acid or a salt thereof, even more preferably disodium cromoglycate, is slowly added until completely dissolved, while maintaining the temperature between 35° C. and 45° C., preferably 40° C. (+/−2° C.).

Next, the dissolution of the compound of formula (I) is added, under stirring and maintaining the temperature between 35° C. and 45° C., preferably 40° C. (+/−2° C.), to the emulsion prepared parallel thereto. The mixture is homogenised and the temperature is maintained between 35° C. and 45° C., preferably 40° C. (+/−2° C.).

Next, the Croton lechleri resin is added to the homogenised mixture. In those cases where aloe vera gel and/or rosehip oil are used, the following are added to the cooled mixture, one by one and preferably in the following order: i) Croton lechleri resin; ii) aloe vera gel; iii) rosehip oil. In both cases, any other component that may be sensitive at high temperatures is also added. Next, in all cases, the mixture obtained is homogenised to obtain the composition of the invention. During this addition and homogenisation phase the temperature is also maintained between 35° C. and 45° C., preferably 40° C. (+/−2° C.)

Lastly, it is verified that viscosity and pH are those desired. If not, the viscosity can be modified using common techniques in the field of the invention. Furthermore, if necessary, the pH of the composition can also be adjusted using common in the field of the invention, for example by adding citric acid, hydrochloric acid or sodium hydroxide.

Once the aforementioned parameters are verified and, if necessary, adjusted, the composition is ready for packaging.

### Use

The combinations of the invention, or the compositions comprising a combination according to the present invention, are useful as a pharmaceutical composition, preferably dermatological, for the treatment or prevention of dermatitis.

It has also been observed that the combinations of the invention are capable of regulating different molecular markers associated with dermatitis. Specifically, it was observed that the combinations of the invention induce the synthesis of the neuroprotective docosanoids Neuroprotectin D1 and its isomers; reduce the production of the PAR2 pruritus markers (receptor activated by protease 2) and TRPV1 and 4 (vanilloid transient potential receptor 1 and 4); and reduce the production of COX-2 inflammation markers (cyclooxygenase-2) and TNF-alpha (tumor necrosis factor alpha). In particular, these biomolecular effects are observed or are particularly pronounced when the proportion by weight between the weight of the compound of formula (I) (or a salt or solvate thereof), preferably of cromoglicic acid (or a salt or solvate thereof), preferably of disodium cromoglycate (or a solvate thereof); and weight B is preferably 2.5:2.045 or higher, more preferably 4.5:2.045 or higher; or when the weight of compound of formula (I) (or a salt or solvate thereof), preferably of cromoglicic acid (or a salt or solvate thereof), is preferably 2.5% or higher with respect to the total weight of the composition, preferably 4.5% or higher.

Therefore, in a preferred embodiment, the present invention is aimed at the combinations of the present invention for use in the treatment or prevention of dermatitis through the modulation of PAR2, more specifically through the reduction of PAR2 expression/levels. The correlation between the PAR2 marker and dermatitis is widely documented in the state of the art, for example in Zhu et al., Int Immunopharmacol, 2015, 28:507-512, in Zhu et al., Int Immunopharmacol 2009, 9:1332-1336; or in Carvalho et al., Exp Dermatol, 2010, 19:117-122. Thus, in one embodiment, the combinations of the present invention are useful in the treatment or prevention of itching and/or inflammation associated with dermatitis, and in a more particular embodiment, the combinations of the present invention are useful in the treatment or prevention of itching and/or inflammation associated with the dermatitis through the modulation of, more specifically, reduction of PAR2 expression/levels.

Also, in another preferred embodiment, the present invention is aimed at the combinations of the present invention for use in the treatment or prevention of dermatitis through the modulation of TRPV4, more specifically through the reduction of TRPV4 expression/levels. The correlation between the TRPV4 marker and dermatitis is widely documented in the state of the art, for example in White et al., Physiol Rev, 2016, 96:911-973; or in Poole et al., J Biol Chem, 2013, 22, 288:5790-5802. Thus, in a more particular embodiment, the combinations of the present invention are useful in the treatment or prevention of itching and/or inflammation associated with dermatitis through the modulation of, more specifically, reduction of TRPV4 expression/levels.

In a more particular embodiment, the combinations of the present invention are useful in the treatment or prevention of itching and/or inflammation associated with dermatitis through the modulation of, more specifically, reduction of PAR2 and TRPV4 expression/levels.

Also, in another preferred embodiment, the present invention is aimed at the combinations of the present invention for use thereof in the treatment or prevention of dermatitis through the modulation of TNF-alpha, more specifically through the reduction of TNF-alpha expression/levels. The correlation between the TNF-alpha marker and dermatitis is widely documented in the state of the art, for example in Dinarello, Chest, 2000, August 118(2):503-8; or in Danso et al. J Invest Dermatol, 2014, ul 134(7):1941-50. Thus, in a more particular embodiment, the combinations of the present invention are useful in the treatment or prevention of inflammation associated with dermatitis through the modulation of, more specifically, reduction of TNF-alpha expression/levels.

In one embodiment, itching or inflammation associated with dermatitis are, respectively, itching or inflammation in subjects suffering from dermatitis (treatment) or, respectively, itching or inflammation that the subjects would suffer with the onset of dermatitis (prevention).

Additionally, as mentioned earlier, it was discovered that the ingredients of the combination unexpectedly act synergistically. Specifically, it was observed that the compound of formula (I) (or a salt or solvate thereof), preferably cromoglicic acid (or a salt or solvate thereof), preferably disodium cromoglycate (or a solvate thereof), acts synergistically with the other constituents of the combination, particularly with Croton lechleri resin and panthenol. In this connection, due to the synergistic effect a smaller dose of the compound of formula (I) may be required, or of Croton lechleri and panthenol (in comparison with the sum of the amounts used in monotherapy) to obtain the same or even greater effectiveness than with the sum of the effectiveness of the respective monotherapies, and the potential toxic side effects observable in monotherapy can be reduced or even prevented. Alternatively, if the dose of the compound of formula (I) and of Croton lechleri resin and panthenol is the same as when procured in monotherapy, an increase in the effectiveness of the combination greater than the sum of the effectiveness of the monotherapies can be expected.

Therefore, in one embodiment, the combinations of the invention, or the compositions of the invention, are synergistic combinations or compositions.

The invention also relates to a compound of formula (I) (or a salt or solvate thereof), preferably cromoglicic acid (or a salt or solvate thereof), preferably disodium cromoglycate (or a solvate thereof), for use in the treatment of dermatitis, which comprises administering a therapeutically effective amount of the compound of formula (I), or of the salt or solvate thereof, in combination, preferably synergistic combination, with a therapeutically effective amount of Croton lechleri resin and panthenol. The invention also relates to a compound of formula (I) (or a salt or solvate thereof), preferably cromoglicic acid (or a salt or solvate thereof), preferably disodium cromoglycate (or a solvate thereof), for use in the treatment of dermatitis, which comprises administering a therapeutically effective amount of the compound of formula (I), or of the salt or solvate thereof, in combination, preferably in synergistic combination, with a therapeutically effective amount of Croton lechleri resin and panthenol, and additionally with a therapeutically effective amount of aloe vera gel and/or rosehip oil.

In a preferred embodiment, the compositions of the invention are applied topically, preferably to the skin. The topical application may be in affected areas or unaffected areas (prophylaxis).

In the context of the present invention, the term dermatitis generally encompasses any inflammatory skin reaction.

In a more particular embodiment, the dermatitis refers or is related to atopic dermatitis, contact dermatitis, seborrheic dermatitis, dermatitis herpetiformis, stasis dermatitis, neurodermatitis, traumatic dermatitis, perioral dermatitis, exfoliative dermatitis, caloric dermatitis (congelationis or ambustionis), X-ray dermatitis, eczema, psoriasis, dermatitis associated with vasculitis, Behcet's Syndrome, pemphigus, hives, urticaria pigmentosa, pyoderma gangrenosum, ulcers, burns, insect stings/bites, herpetic infections, multiple sclerosis (systemic scleroderma), morphoea (circumscribed or localised scleroderma) or dermal nodular fibrosis.

In an even more particular embodiment, dermatitis refers to atopic dermatitis, contact dermatitis, seborrheic dermatitis, dermatitis herpetiformis, stasis dermatitis, neurodermatitis, traumatic dermatitis, eczemas or psoriasis.

In a preferred embodiment, dermatitis refers to atopic dermatitis or contact dermatitis. Even more preferably, the dermatitis is atopic dermatitis.

In one embodiment, the dermatitis is chronic. In another embodiment, the dermatitis is acute.

It is understood that, in the context of the invention, the treatment of the dermatitis is considered both in humans and in animals (veterinary use). In a particular embodiment, the subject affected by dermatitis is an animal, for example a cat, dog or horse. In a preferred embodiment, the subject affected by dermatitis is a human.

In the context of the present invention, it is understood that the amounts of the combinations or the compositions of the invention that are used for the treatment or prophylaxis of dermatitis, or for preparing a drug intended for treatment or prophylaxis of dermatitis, are therapeutically effective amounts. Therapeutically effective amount is understood to be the amount that gives rise to an improvement or prevention detectable in the patient's physiology, i.e. in the patient's dermatitic condition, whereto the combination or composition of the invention is administered or will be administered.

The amounts of the combinations of the invention to be administered, or the compositions comprising a combination according to the present invention, will vary depending on different factors. For example, the dose will vary depending on the specific dermatitis to be treated; in particular, it will vary depending on the severity or amplitude of the affected area. The healthcare expert knows how to consider the different factors involved and determine the specific dose for a certain patient.

In a particular embodiment, the compositions comprising a combination according to the present invention are disposed in containers, for example dispensers, that administer a predetermined amount of composition per pulsation. Preferably, the container administers between 1.0 and 0.1 mL, more specifically between 0.7 and 0.2 mL, even more specifically between 0.45 and 0.55 mL per pulsation. Preferably, the composition contained is in the form of a cream.

The administration regime of the combinations of the invention, or the compositions comprising a combination according to the present invention, will also vary depending on the aforementioned different factors. The healthcare expert knows how to determine a suitable administration regime for a certain patient.

In a particular embodiment, the combinations of the invention, or the compositions comprising a combination according to the present invention, are administered for as long as necessary for the dermatitis to disappear or until it is no longer detectable. In a more specific embodiment, they are administered for one or two months. In another more specific embodiment, they are administered for one, two or three weeks. In another more specific embodiment, they are administered for one to six days, preferably for six to four days, more preferably for five days.

In a particular embodiment, the combinations of the invention, or the compositions comprising a combination according to the present invention, are administered one, two or three times a day; one, two or three times every two days; or one, two or three times per week. Preferably, they are administered twice a day.

In an even more particular embodiment, they are administered twice a day for any of the aforementioned periods, preferably for one or two months.

In another even more particular embodiment, they are administered three times a day for one to six days, preferably for five days.

The administration regime does not have to be the same throughout the treatment, understanding that there may be different administration frequencies; for example, initially an administration can be more frequent and subsequently an administration can be less frequent.

According to the present invention, the constituents of the combination of the invention
a) compound of formula (I) (or a salt or solvate thereof), preferably cromoglicic acid (or a salt or solvate thereof), preferably disodium cromoglycate (or a solvate thereof);
b) Croton lechleri resin;
c) panthenol;
and, where applicable,
d) aloe vera gel; and/or rosehip oil,

can be provided in the same pharmaceutical composition, as described earlier, or in separate compositions for administration at the same or different times. If the aforementioned constituents are administered at different times, they must be administered sufficiently close in time, for example, they must be administered during the same day, or in two days' time, in order to ensure that the therapeutic effectiveness and, above all, the synergistic response, are maintained

When the constituents are administered in separate compositions, these compositions are prepared and are of the type as described earlier.

Furthermore, the combinations of the invention, or the compositions comprising a combination according to the present invention, can be used cosmetically to treat or reduce the effects of dermatitis, for example, as skin regenerators or moisturisers or antioxidants. In one embodiment, cosmetic use is made once the inflammation has been controlled (the inflammation disappears) but the effects thereof continue (for example, injuries). Cosmetic use comprises applying a cosmetically effective quantity of the composition or combination of the invention. It is understood that, in the context of the invention, these cosmetic uses are envisaged both in humans and in animals. In a particular embodiment, the subject is an animal, for example a cat, dog or horse. In a preferred embodiment, the subject is a human.

### EXAMPLES

### Example 1: Preparation of the Composition 5% Disodium Cromoglycate (5% DSCG)

The indicated percentages correspond to the percentage by weight of each ingredient in the final composition obtained.

In a reactor equipped with previously sterilised stirring equipment, water (q.s.), disodium EDTA (0.2%), propylene glycol (5%), glycerine (3%) and D-panthenol (2%) are added, mixed together and heated at 75° C.-80° C. In another separately conditioned receptacle, Steareth-2 (3.5%), stearyl alcohol (3.5%), Steareth-21 (3%), cetearyl ethylhexanoate (5.7%), paraffin oil (3%), beeswax (0.5%), dimethicone (0.8%), phenoxyethanol (0.67%), stearoxydimethicone (0.5%), cocoglycerides (0.5%), isopropyl myristate (0.3%), stearic acid (0.2%), ethylhexylglycerine (0.075%) are added, mixed together and heated at 75° C.-80° C. Next, an emulsion is prepared from the lipophile mass and the hydrophilic components with appropriate stirring. Lastly, the emulsion obtained is cooled to 40° C. (+/−2° C.).

Parallel to the emulsion method, water (10%) is heated at a temperature of 40° C. (+/−2° C.) in a different, similarly conditioned receptacle, after which disodium cromoglycate (5%) is slowly added, avoiding the formation of lumps, until completely dissolved. A gel is formed.

Next, the aqueous disodium cromoglycate solution is added, under stirring, to the emulsion prepared parallel thereto. The mixture is homogenised. During this addition and homogenisation phase a temperature of 40° C. is maintained (+/−2° C.).

Next, Croton lechleri resin (0.045%), propanediol (1.48%), aloe vera gel (concentrated 10:1) (3%), rosehip oil (0.5%) and imidazolidinyl urea (0.25%) are added one by one and in the indicated order to the homogenised mixture to obtain the composition of the invention. During this addition and homogenisation phase a temperature of 40° C. is maintained (+/−2° C.). Lastly, pH is adjusted by addition of NaOH (0.5%).

### Example 2: In Vivo Study of the Treatment of Dermatitis

The following formulations were assayed in a mouse dermatitis model:

| Group of mice | Formulation applied |
|---|---|
| C | none |
| DC | none |
| D | 5% DSCG |
| D | 2% DSCG |
| D | Non-DSCG |
| D | 2% DSCG (non-CL/P) |

wherein

C=control (mice without dermatitis);
DC=dermatitis control (sensitised mice that do not receive treatment);
D=dermatitis (sensitised mice that receive treatment).
The 5% DSCG formulation is the composition prepared according to Example 1.

The 2% DSCG formulation is a composition prepared following the preparation method of Example 1 but wherein an amount of disodium cromoglycate representing only 2% by weight with respect to the total weight of the composition was added.

The Non-DSCG formulation is a comparative composition prepared following the preparation method of Example 1 but wherein the disodium cromoglycate addition stage was omitted, such that the composition does not comprise disodium cromoglycate.

The 2% DSCG (non-CL/P) formulation is a composition prepared following the preparation method of Example 1 but wherein the Croton lechleri resin and panthenol addition stage was omitted, and wherein an amount of disodium cromoglycate representing only 2% by weight with respect to the total weight of the composition was added.

### Animal Dermatitis Model

The following sensitisation protocol for inducing atopic dermatitis in mice was followed. 2.4-dinotrofluorobenzene (DNFB) was applied to the outer surface of both ears of BALB/c strain mice every two days. After 19 days the application of DNFB ceased. It was observed that, during the sensitisation phase, ear thickness increased from an initial 0.20 mm to 0.48 mm (an increase of 0.28 mm or 100%) in 18.

### Treatment

As soon as the application of DNFB ceased, the application of the different formulations (creams) began. Two groups of mice served as controls: a first “C” group that was not sensitised or treated with a formulation and a second “CD” group that served as a dermatitis control, which was sensitised but was not treated with a formulation.

The assayed formulations (5% DSCG, 2% DSCG, Non-DSCG, 2% DSCG (non-CL/P)) were applied to the ears of a respective group of mice for seven days, and the ears were collected.

All the groups of mice consisted of six mice (12 ears).

### Results

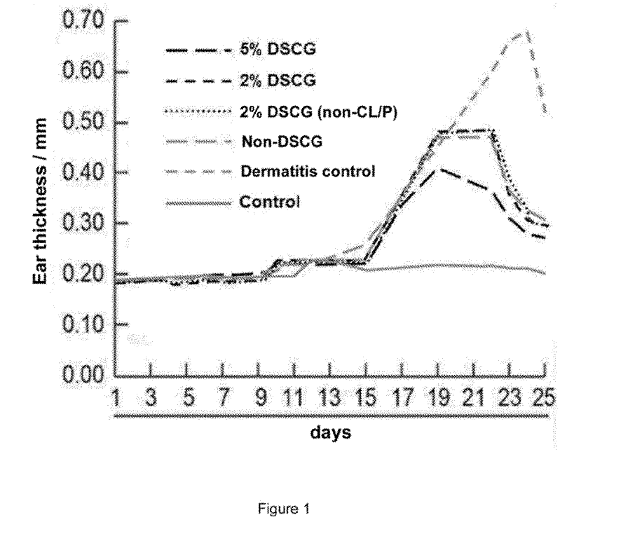

As might be expected, no significant variation was observed in the C group in ear thickness (FIG. 1). In the DC group, after ceasing the application of DNFB, ear thickness continued to increase for five days, reaching 0.68 mm, after which the thickness began to decrease. In the other cases wherein, after ceasing the application of DNFB, one of the assayed formulations was applied, two different behaviours were observed. In the groups treated with the formulations 2% DSCG, Non-DSCG and 2% DSCG (non-CL/P), ear thickness stopped increasing and remained relatively constant for three days, observing a decrease in ear thickness in the following three days until reaching an ear thickness of approximately 0.30 mm (a reduction of 0.18 mm or 64%) after six days. In the group treated with formulation 5% DSCG, a surprising synergistic effect was observed that implied a decrease in ear thickness from the moment in which the formulation was applied and a faster and accentuated decrease, reaching a thickness of 0.27 mm (a decrease of 0.21 mm or 75%) after six days.

### Example 3: In Vivo Study II of the Treatment of Dermatitis

### Animal Dermatitis Model

The following sensitisation protocol for inducing atopic dermatitis in mice was followed. 2,4-dinotrofluorobenzene (DNFB) was applied to the outer surface of both ears of BALB/c strain mice. When the mouse ear reached 0.55 mm the application of DNFB ceased.

### Treatment

As soon as the application of DNFB ceased, the application of the formulations (creams) 5% DSCG and 2% DSCG began. Two groups of mice served as controls: a first “C” group that was not sensitised or treated with a formulation; and a second “CD” group that served as a dermatitis control, which was sensitised but not treated with a formulation.

The assayed formulations (5% DSCG, 2% DSCG) were administered, respectively, to two groups of mice for nine days and the ears were collected.

All the groups of mice consisted of four mice (eight ears).

### Results

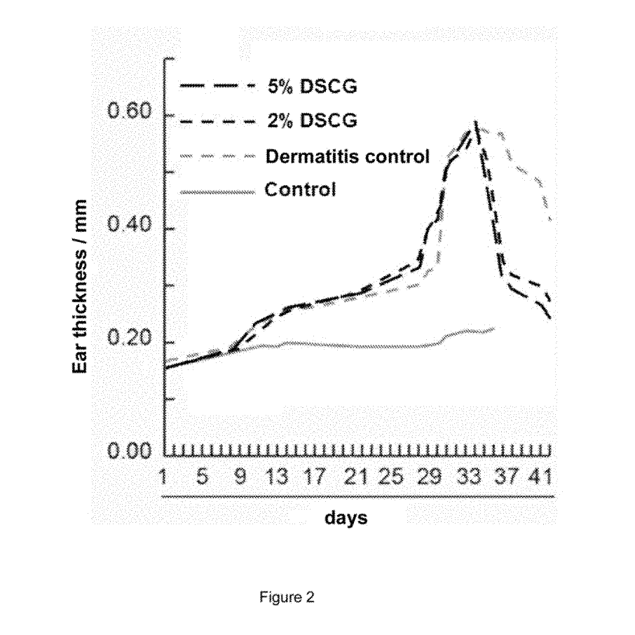

As might be expected, in the C group no significant change in ear thickness was observed (FIG. 2). In the groups treated with formulations, ear thickness decreased rapidly after three days of treatment with the formulations, while in the DC group ear thickness was maintained at around 0.57 mm. After eight days of treatment, ear thickness in the 5% DSCG group decreased to 0.25 mm; in the 2% DSCG group it decreased to 0.28 mm; and in the DC group it decreased to 0.42 mm.

### Example 4: In Vivo Study III of the Treatment of Dermatitis

A similar protocol to that of Example 2 was followed, wherein the sensitisation phase lasted seven days (first week), and the compositions were applied during the second week. Once again, similar results were observed, especially in relation to the synergistic activity of the composition 5% DSCG on the dermatitis. The results observed for the different compositions assayed are shown numerically below:

| Composition | Decrease in ear thickness |
|---|---|
| Non-DSCG | 7 mm |
| 2% DSCG (non-CL/P) | 4 mm |
| 5% DSCG | 20 mm |

The theoretical additive activity for formulation 5% DSCG was a decrease of 17 mm (7 mm attributable to Croton lechleri resin and panthenol, and 10 mm attributable to DSCG [2.5×4 mm]). However, it was unexpectedly observed that the decrease was greater, specifically 20 mm, evidencing the synergistic effect of using the combinations of the present invention.

### Example 5: In Vivo Study of the Prevention of Dermatitis

The assay of example 2 was followed with modifications. Specifically, the formulations were applied parallel to the application of 2,4-dinotrofluorobenzene (DNFB). It was observed that formulations 5% DSCG and 2% DSCG successfully prevented ear thickening compared to the dermatitis control group. Also, once again, it was observed that the action of formulation 5% DSCG acts synergistically. It was observed that formulation 5% DSCG has great capacity to prevent pruritus and inflammation.

### Example 6: Study of PAR2, TRPV4 and TNF-Alpha Levels

The tissue of the ears obtained after the assay of Example 4 was ground and homogenised (glass on glass) at 4° C. using RIPA buffer (Thermo-Fisher Scientific) formed from a mixture of protease and phosphatase inhibitor (Roche Diagnostics, Indianapolis, Ind.). The samples were sonicated and centrifuged for 10 minutes at 14,000 g. The concentration of protein was determined by means of a Bradford assay (Bio-Rad, Hercules, Calif.). The surfactant was used for electrophoresis in polyacrylamide gel with sodium dodecylsulfate (SDS-PAGE) and the pellet was stored at 20° C. Large, equal amounts of boiled protein were diluted (20 μg per lane) with SDS buffer, loaded in pre-moulded BioRad Criterion gels (4-12%) for SDS-PAGE (BioRad, Hercules, Calif.) and subject to electrophoresis at 125 V for 1.5 hours in ice. The proteins were studied in polyvinylidene fluoride (PVDF) membranes using the BioRad Criterion TransBlot system. The transfer was carried out in a turbo transfer system (BioRad) following the manufacturer's instructions. Gel retention was evaluated by means of Coomassie blue dye (Pierce, Rockford, Ill.). The non-specific bond was blocked for 1 hour at room temperature with SuperBlock blocking solution (Thermo-Fisher Scientific). The membranes were incubated during the night at 4° C. with primary antibodies against PAR2, TRPV4 and TNF-α in TTBS. The bonded primary antibody was detected by means of a secondary antibody linked to HRP at 55, 98 and 17 kDa, respectively. The protein bands were viewed using a Fujifilm LAS-3000 digital scanner following the manufacturer's instructions and quantified using ImageQuant TL software (GE Healthcare). The membranes were separated for 30 minutes, re-assayed with GAPDH (38 kDa, monoclonal mouse IgG) as a load control (EMD Millipore) and detected with mouse anti-IgG HRP (Santa Cruz).

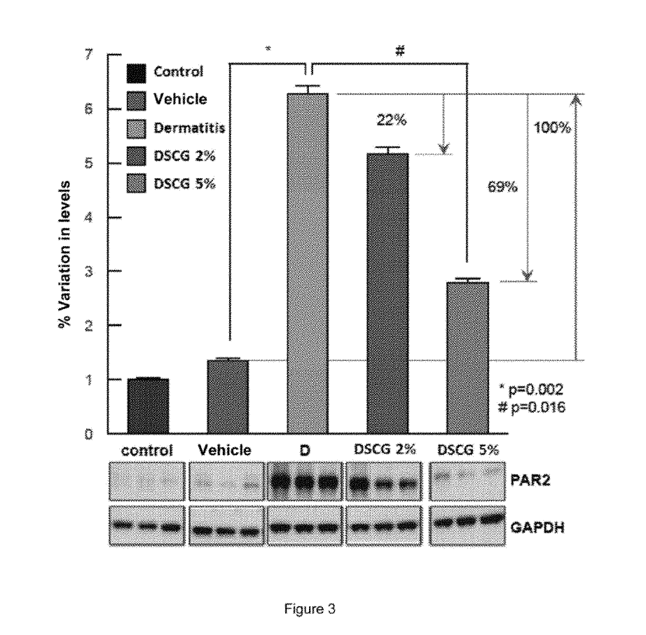

The results are shown in FIGS. 3 (PAR2), 4 (TRPV4) and 5 (TNF-alpha). A clear reduction in the levels of these three markers in the samples derived from the ears were treated with the combinations of the invention, and more particularly with composition DSCG 5%, wherein a synergistic effect is observed once again, evidenced by the decrease in PAR2 levels, by more than three times (from 22% to 69%), due to the increase in DSCG by only 2.5 times (from 2% to 5%).

1. A combination comprising:
a) a compound of formula (I) 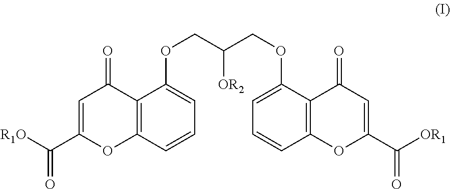
wherein
each R₁, independently, is selected from H or, together with the —O—C(O)— group whereto it is bonded, forms an ester or carboxylic anhydride group; and
R₂ is selected from H or, together with the oxygen atom whereto it is bonded, forms an ether, ester, carbamate or carbonate group;
or a salt or solvate thereof;
b) Croton lechleri resin; and
c) panthenol.

2. The combination, according to claim 1, wherein the compound of formula (I) is cromoglicic acid or a salt or solvate thereof.

3. The combination, according to claim 2, wherein the compound of formula (I) is disodium cromoglycate or a solvate thereof.

4. The combination, according to claim 1, wherein the proportion by weight of the compound of formula (I) with respect to the combined weight of Croton lechleri resin and panthenol is 2.5:2.045 or higher.

5. The combination, according to claim 1, further comprising: d) aloe vera gel and/or rosehip oil.

6. A pharmaceutical composition comprising:
a) a combination as defined in claim 1; and
b) a pharmaceutically acceptable carrier.

7. The pharmaceutical composition, according to claim 6, wherein the compound of formula (I) comprised in the combination is present in a weight between 2.5% and 10% with respect to the total weight of the pharmaceutical composition.

8. The pharmaceutical composition, according to claim 7, wherein the compound of formula (I) comprised in the combination is present in a weight between 4.5% and 5.5% with respect to the total weight of the pharmaceutical composition.

9. The pharmaceutical composition, according to claim 6, which additionally comprises at least one active ingredient selected from corticosteroids, antihistamines, antiallergens and antibacterials.

10. The pharmaceutical composition, according to claim 6, wherein the pharmaceutical composition is an emulsion of the oil in water type.

11. The pharmaceutical composition, according to claim 6, wherein the pharmaceutical composition is a cream, lotion or milk.

12.-17. (canceled)

18. A method for preparing an emulsion comprising the combination of claim 1, the method comprising:
preparing a carrier by combining the panthenol with water and glycerine, heating, adding a lipophilic mass, and stirring;
separately heating water and adding the compound of formula (I) to form a dissolution of the compound of formula (I);
adding the dissolution of the compound of formula (I) under stirring to the carrier and homogenizing to form a mixture;
adding the mixture to the Croton lechleri resin to the homogenized mixture; and
determining a viscosity and pH for the homogenized mixture and modifying, if necessary.

19. A method for therapeutic treatment or prevention of dermatitis, itching and/or inflammation associated with dermatitis, and atopic dermatitis, comprising:
providing an emulsion comprising the combination of claim 1; and
applying topically a therapeutically effective amount of the emulsion to an area of skin to be treated.

20. A method for cosmetic treatment of dermatitis, itching and/or inflammation associated with dermatitis, and atopic dermatitis, comprising:
providing an emulsion comprising the combination of claim 1; and
applying topically a cosmetically effective amount of the emulsion to an area of skin to be treated.
